(19) United States
(12) Patent Application Publication
(10) Pub. No.: US 2011/0125109 A1
(43) Pub. Date: May 26, 2011

(54) WOUND STICK

(76) Inventor: Erik B. Young, Annapolis, MD (US)

(21) Appl. No.: 12/943,396

(22) Filed: Nov. 10, 2010

Related U.S. Application Data
(60) Provisional application No. 61/272,847, filed on Nov. 10, 2009.

(51) Int. Cl. A61M 31/00; A61K 9/00; A61P 7/04

(52) U.S. Cl. 604/286; 424/400

(57) ABSTRACT

A device for introducing a haemostatic agent into a penetrating wound. The device includes an elongate introducing member and a haemostatic agent applied to the elongate introducing member. The haemostatic agent may be applied to the introducing member using a flexible membrane, configured to be rolled around the elongate introducing member, a biocompatible foam, or a gel.

### CROSS REFERENCE TO RELATED APPLICATIONS

The present application claims priority to U.S. provisional Patent Application Ser. No. 61/272,847, to Erik B. Young, entitled “WOUND STICK”, which is incorporated, in its entirety, herein by reference.

### FIELD OF THE INVENTION

The present invention relates to a method and apparatus for the initial treatment of a penetrating wound. More particularly, the present invention relates to a method and apparatus for introducing a haemostatic agent into a penetrating wound.

### BACKGROUND OF THE INVENTION

Many different types of bandages and mechanical devices are known in the art for controlling hemorrhage. Mechanical devices used to control hemorrhage include (i) tourniquets that compress the soft tissues surrounding the long bones in a limb thereby compressing the blood vessels providing blood to the area of a wound, (ii) instruments that can be clamped across the large blood vessels in a wound (a “hemostat”), and (iii) pressure dressings that apply direct pressure to areas of bleeding. Another method to control bleeding is through the direct application topical agents to a wound that promote haemostatis (clot formation) referred to herein as haemostatic agents.

### SUMMARY OF THE INVENTION

The methods currently being employed to treat hemorrhage have many drawbacks. The prolonged use of a tourniquet is associated with post treatment complications, the application of a hemostat to a bleeding vessel is very difficult outside of an operating room, and the application of a pressure dressing to a wound frequently fails to control hemorrhagic bleeding. The treatment of hemorrhage in a penetrating wound is further complicated by the fact that the wound tract is narrow and dressings, instruments, or haemostatic agents can not be applied to the area of the bleeding.

In view of the foregoing and other exemplary problems, drawbacks, and disadvantages of the conventional methods and structures, an exemplary feature of the present invention is to provide an apparatus and method for initial treatment of a penetrating wound, which introduces a haemostatic agent into the penetrating wound.

In a first exemplary, non-limiting aspect of the present invention, a device includes an elongate introducing member and a haemostatic agent applied to the elongate introducing member.

In a second exemplary, non-limiting aspect of the present invention, a device includes an elongate introducing member, a flexible and permeable or perforated membrane configured to be rolled around the elongate introducing member, and a haemostatic agent rolled within or disposed on the membrane.

In a third exemplary, non-limiting aspect of the present invention, a device includes an elongate introducing member and a material surrounding the elongate introducing member, the material being impregnated with a haemostatic agent.

According to certain exemplary aspects of the present invention, the device may be used for initial treatment of straight (or nearly straight) penetrating wounds of a nearly uniform diameter that result from, for example, ballistic or other penetrating injuries. The device may be configured to introduce a haemostatic agent into the wound (e.g., the full extent of the wound). In the context of the claimed invention, as will be described in further detail below, the haemostatic agent may be defined as a coagulant or a combination of coagulants.

The device may be used by first responders who suspect that a wound represents a significant risk of hemorrhage that cannot be controlled by the use of a hemostat or a pressure dressing. According to an exemplary aspect of the present invention, hemorrhage originating in a penetrating wound can be controlled by a first responder by using the device alone or in conjunction with a tourniquet for a short duration.

These and other exemplary features and advantages of the present invention will become clear from the following description with reference to the accompanying drawings.

### BRIEF DESCRIPTION OF THE DRAWINGS

The foregoing and other exemplary purposes, aspects and advantages will be better understood from the following detailed description of an exemplary embodiment of the invention with reference to the drawings, in which:

FIG. 1 illustrates a device 100 according to a first exemplary embodiment of the present invention;

FIG. 2 illustrates a cross sectional view of the device 100;

FIG. 3 illustrates a cross sectional view of a first end of the device 100;

FIG. 4 illustrates an expanded view of a membrane of the device 100; and

FIG. 5 illustrates a device 200 according to another exemplary embodiment of the present invention.

### DETAILED DESCRIPTION OF CERTAIN EXEMPLARY EMBODIMENTS OF THE PRESENT INVENTION

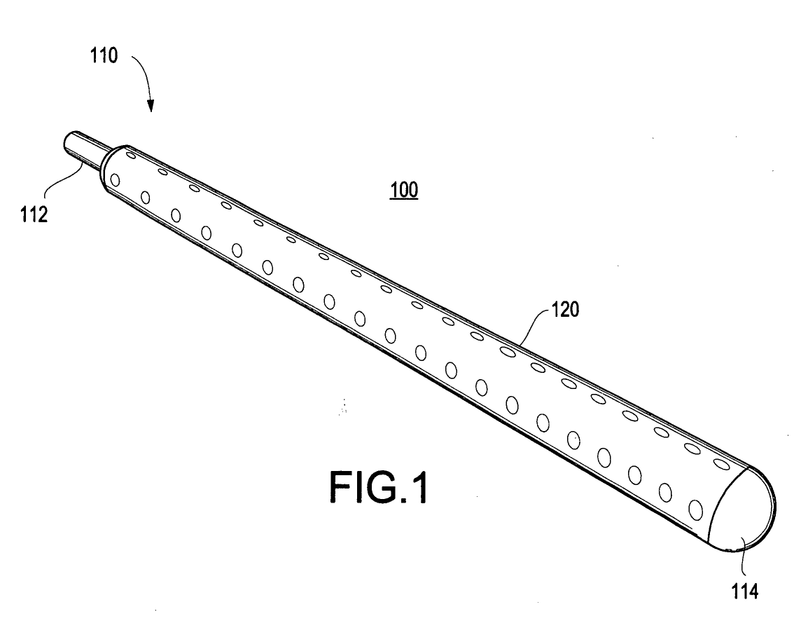

Referring now to the drawings, and more particularly to FIGS. 1-5, there are shown exemplary, non-limiting embodiments of the method and structures according to the present invention.

The device includes a rigid or semi-rigid device for the initial treatment of straight (or nearly straight) penetrating wounds of a nearly uniform diameter that result from, for example, ballistic or other penetrating wounds.

FIG. 1 illustrates a device 100 according to a first, exemplary, non-limiting embodiment of the present invention. The device includes an introducing member 110. The introducing member includes a rigid or semi-rigid, elongate main body having a handle 112 disposed at a first end and a smooth tapered 114 end at a second end. The smooth tapered end 114 is configured to be introduced into the wound tract.

The introducing member 110, including the smooth tapered end 114 (e.g., rounded end) may be radio opaque. Accordingly, the location of the device 100 can be related to vital anatomical structure using radiological studies.

The introducing member 110 shall be long enough so that a wound traversing a limb can be treated. Additionally, a user of the device should be able to shorten the device with bandage scissors, if necessary.

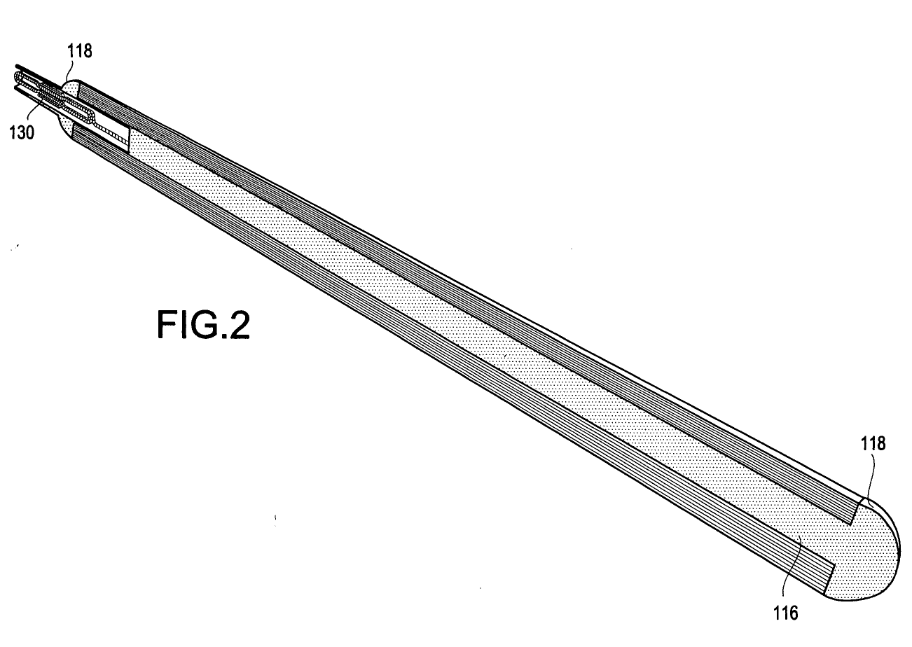

Referring to FIG. 2, the introducing member 110 includes an elongate center shaft 116 and a flange portion 118 at each end thereof.

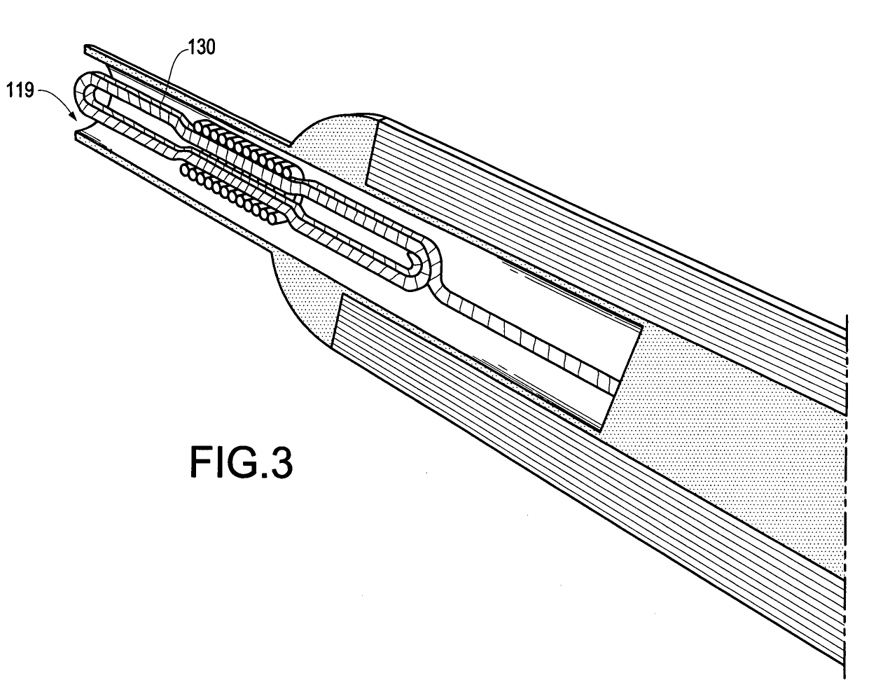

Referring to FIG. 3, the handle 112 includes an opening 119 at an end thereof. The handle 112 and the central shaft 116 include a hollow portion configured to receive a string 130. The string 130 allows a user of the device to easily remove the device 100 from the wound.

Turning back to FIG. 1, the device 100 includes a flexible membrane 120 disposed on the introducing member 110. As is illustrated in FIG. 2, the flexible membrane 120 may be configured to be rolled around the central shaft 116 of the introducing member 110. The flexible membrane 120 is held in place between the flange portions 118 at each end of the central shaft 116.

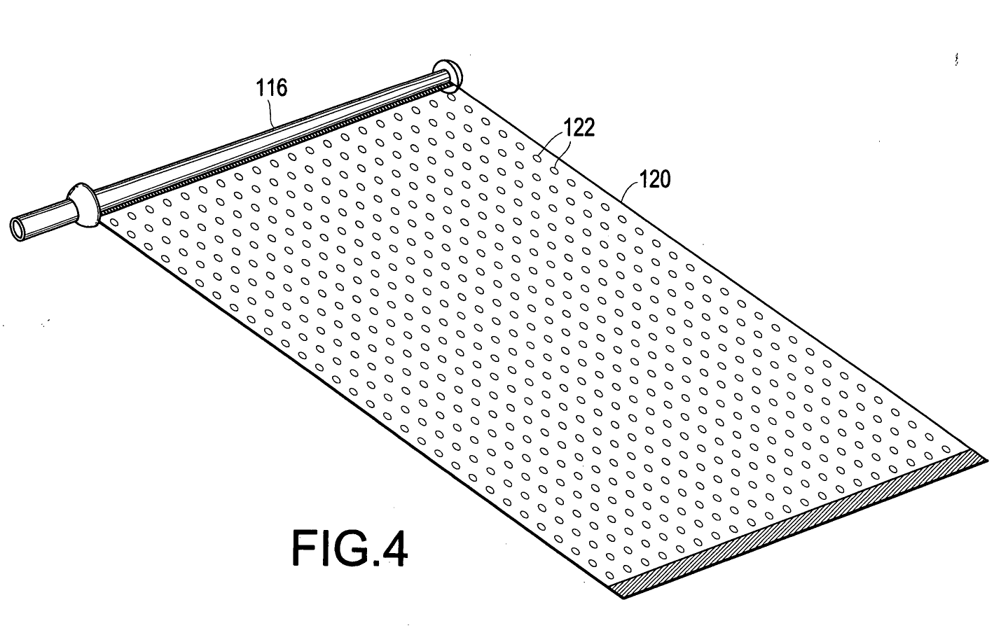

The flexible membrane 120, in its unrolled state, as illustrated in FIG. 4, has a top surface and a bottom surface. A haemostatic agent is placed between the rolled layers of the membrane. The haemostatic agent may be chemically or physically bonded to one or both surfaces of the membrane, prior to rolling.

The haemostatic agent may include a coagulant or a combination of several coagulants. Additionally, the haemostatic agent may include an absorbent agent that is expandable in the presence of moisture in combination with the coagulant or combination of coagulants. If the haemostatic agent includes an expanding absorbent, then the membrane can be configured to be stretchable so that the device can expand within the wound.

FIG. 4 illustrates the device 100 in a state in which the flexible membrane 120 is unrolled. The flexible membrane 120 includes a plurality of small or microscopic perforations. The size and number of perforations depends on the type of haemostatic agent used. The perforations 122 allow the haemostatic agent to diffuse into the wound. Additionally, the perforations 122 allow the blood and plasma in the wound to come into contact with the haemostatic agent. In the event the haemostatic mixture contains particles, the perforations/holes 122 in the membrane 120 can be smaller than the particle size to prevent their extrusion into the wound.

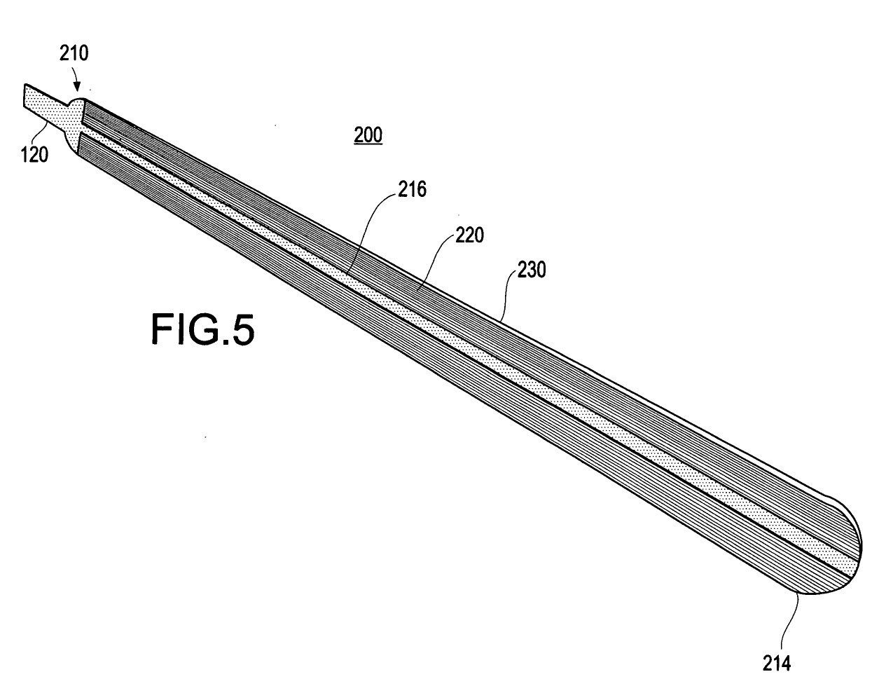

FIG. 5 illustrates several other exemplary, non-limiting embodiments of the present invention. Similar to the device discussed above, with respect to FIGS. 1-4, the device 200 illustrated in FIG. 5 includes an elongate introducing member 210 having a central shaft 216, a handle 212 disposed at a first end of the central shaft 212, and a smooth tapered end 214 disposed at a second end of the central shaft 216.

Instead of the flexible membrane of the previous embodiments, the exemplary, non-limiting embodiments illustrated in FIG. 5 include the central shaft 216 of the introducing member 216 surrounded by a material 220 that contains a haemostatic agent.

The material containing the haemostatic agent may comprise any semi-rigid material that can be impregnated with the haemostatic agent or that can be contained within a sheath surrounding the central shaft 216 and the material 220 (discussed below). Additionally, the material 220 containing the haemostatic agent may be made from a variety of materials that can release the haemostatic agent once the device is placed in the wound.

According to certain exemplary embodiments of the present invention, the material 220 comprises a compressed, biocompatible foam (e.g., spongy foam) cylinder 220 surrounding the central shaft 216 of the introducing member 216. The foam cylinder 220 is impregnated with a haemostatic agent. Specifically, the foam cylinder 220 may be impregnated with a haemostatic agent (e.g., coagulant or combination of several coagulants) in either a dried or liquid state.

The device 200 includes a sheath 230, which surrounds the foam cylinder and keeps the foam compressed. The sheath 230 is removed from the device once the device 200 is placed in the wound. The haemostatic agent is activated when the removable sheath 230 is removed from the device 200 and the compressed foam expands to its original shape and perhaps further as the foam absorbs moisture.

According to other exemplary embodiments of the present invention, the material 220 comprises a semi-rigid gel 220 surrounding the central shaft 216 of the introducing member 216. The semi-rigid gel 220 contains a haemostatic agent.

The device 200 includes a sheath 230, which surrounds the semi-rigid gel 220, which maintains the elongated shape of the gel. The sheath 230 is removed from the device once the device 200 is placed in the wound. The haemostatic agent is activated when the removable sheath 230 is removed from the device 200.

The present invention may be used by first responders who suspect that a wound represents a significant risk of hemorrhage that cannot be controlled by a conventional pressure dressing and can be controlled with this device without the use of a tourniquet. In some cases, however, the penetrating wound of a limb might be initially treated with both a tourniquet and the device of the exemplary embodiments of the present invention and once coagulation has been promoted in the wound, the tourniquet may be removed.

While the invention has been described in terms of several exemplary embodiments, those skilled in the art will recognize that the invention can be practiced with modification within the spirit and scope of the appended claims.

Further, it is noted that, Applicant's intent is to encompass equivalents of all claim elements, even if amended later during prosecution.

What is claimed is:

1. A device, comprising:
an elongate introducing member; and
a haemostatic agent applied to the elongate introducing member.

2. The device according to claim 1, wherein the haemostatic agent comprises a coagulant or a combination of several coagulants.

3. The device according to claim 2, wherein the haemostatic agent further comprises an absorbent agent.

4. The device according to claim 1, wherein the elongate introducing member comprises an elongate center shaft, the haemostatic agent being disposed such that the haemostatic agent surrounds the elongate center shaft.

5. The device according to claim 1, wherein the elongate introducing member comprises a smooth tapered end.

6. A device, comprising:
an elongate introducing member;
a flexible and permeable or perforated membrane configured to be rolled around the elongate introducing member; and
a haemostatic agent rolled within or disposed on the membrane.

7. The device according to claim 6, wherein the flexible and permeable or perforated membrane comprises a thin film having a plurality of perforations disposed in an array on the flexible and permeable or perforated membrane.

8. The device according to claim 6, wherein the haemostatic agent is disposed on at least one surface of the flexible and permeable or perforated membrane.

9. The device according to claim 6, wherein the haemostatic agent is disposed between layers of the flexible and permeable or perforated membrane when the membrane is rolled around the elongate introducing member.

10. The device according to claim 6, wherein the haemostatic agent is chemically or physically bound to at least surface of the flexible and permeable or perforated membrane.

11. The device according to claim 6, wherein the haemostatic agent comprises a coagulant or a combination of several coagulants.

12. The device according to claim 11, wherein the haemostatic agent further comprises an absorbent agent.

13. A device, comprising:
an elongate introducing member; and
a material surrounding the elongate introducing member, the material being impregnated with a haemostatic agent.

14. The device according to claim 13, wherein the material comprises a compressed, biocompatible, foam cylinder surrounding the elongate introducing member.

15. The device according to claim 13, wherein the material comprises a semi-rigid gel surrounding the elongate introducing member.

16. The device according to claim 13, further comprising a removable sheath disposed around the material.

17. The device according to claim 13, wherein the haemostatic agent comprises a coagulant or a combination of several coagulants.

18. The device according to claim 17, wherein the haemostatic agent further comprises an absorbent agent.
